(19) United States
(12) Patent Application Publication
(10) Pub. No.: US 2024/0033523 A1
(43) Pub. Date: Feb. 1, 2024

(54) NEUROSTIMULATION SYSTEM

(71) Applicant: ADVANCED NEUROMODULATION SYSTEMS, INC., PLANO, TX (US)

(72) Inventor: Daran DeShazo, Lewisville, TX (US)

(21) Appl. No.: 18/342,378

(22) Filed: Jun. 27, 2023

Related U.S. Application Data
(60) Provisional application No. 63/369,784, filed on Jul. 29, 2022.

(51) Int. Cl. A61N 1/36

(52) U.S. Cl. CPC A61N 1/36139; A61N 1/36125

(57) ABSTRACT

Provided is an implantable pulse generator (IPG) for providing electrical pulses to a patient during neurostimulation therapy. The IPG includes a controller for controlling operations of the IPG, a battery for powering the IPG, a therapeutic stimulation circuitry for generating electrical pulses to stimulate neural tissue of the patient, and internal diagnostic circuitry for digitizing device signals to control operations of the IPG. The internal diagnostic circuitry includes a multi-stage comparator including a pre-amplifier configured to receive an analog input signal and convert the analog input signal into a first analog output signal and second analog output signal. The multi-stage comparator also including a first latch circuit with a first receiving device that receives the first analog output signal, a second receiving device that receives the second analog output signal, and configured to convert the first analog output signal and second analog output signal into a digital output signal.

### CROSS-REFERENCE TO RELATED APPLICATIONS

This application claims the benefit of U.S. Provisional Application No. 63/369,784 filed Jul. 29, 2022, titled “NEUROSTIMULATION SYSTEM”, the subject matter of which is hereby incorporated by reference in its entirety.

### BACKGROUND

Embodiments of the present disclosure generally concern neuromodulation, or neurostimulation, systems, and improvements thereof.

Implantable medical devices have improved how medical care is provided to patients with certain types of chronic illnesses and disorders. For example, implantable cardiac devices improve cardiac function in patients with heart disease thereby raising quality of life and reducing morality rates. Implantable neurostimulator can provide pain reduction for chronic pain patients and reduce motor difficulties in patients with Parkinson's disease and other movement disorders. A variety of other medical devices are proposed and are in development to treat other disorders in a wide range of patients.

Neural activity in the brain can be influenced by electrical energy that Is supplied from a stimulation pulse generator or other waveform generator, Various patient perceptions and/or neural functions can be promoted or disrupted by applying an electrical or magnetic signal to the brain. Medical researchers and clinicians have attempted to manage various neurological conditions using electrical or magnetic stimulation signals to control or affect brain functions. For example, Deep Brain Stimulation (DBS) may reduce some of the symptoms associated with Parkinson's Disease, which results in movement or muscle control problems and is debilitating to a great number of individuals worldwide.

A stimulation system pulse generator may be provided in various configurations, such as an implanted pulse generator (IPG). A typical IPG system configuration comprises of a surgically implanted, internally-powered pulse generator and multi-electrode lead. The implanted pulse generator may commonly be encased in a hermetically sealed housing and surgically implanted, for example, in a subclavicular, upper chest, or lower back location. An electrode assembly may be implanted to deliver stimulation signals to a stimulation site. The electrode assembly is coupled to the pulse generator via biocompatible and insulated lead wires. A power source, such as a battery, is contained within the housing of the pulse generator.

Brain anatomy typically requires precise targeting of tissue for stimulation by deep brain stimulation systems. For example, deep brain stimulation for Parkinson's disease commonly targets tissue within or dose to the subthalamic nucleus (STN). The STN is a relatively small structure with diverse functions. Stimulation of undesired portions of the STN or immediately surrounding tissue can result in undesired side effects. For example, muscle contraction or muscle tightening may be caused by stimulation of neural tissue that is near the STN. Mood and behavior dysregulation and other psychiatric effects have been reported from undesired stimulation of neural tissue near the STN in Parkinson's patients.

Neuromodulation systems are used for treatment of a wide range of conditions, and can include measurement functions related to monitoring battery voltage, electrode impedance, temperature, and bio-marker sensing for closed-loop control. These analog measurements are generally digitized by an Analog-to-Digital Converter (ADC). Stimulation waveforms as well as some bio-markers require a sample rate that is fast enough that Successive Approximation Register (SAR) or Flash ADC architectures are preferred over lower-bandwidth Sigma-Delta converters. A key component of both the SAR and Flash is a comparator which must accurately compare the analog input to a reference and provide a single-bit binary output to downstream circuits. Still, while using low-voltage digital technology saves area and power in the downstream processing circuits, the analog input signals are often at a high voltage, and incompatible with such technology.

As a result, a need exists for a neuromodulation system that includes and improved analog to digital conversion of signals.

### SUMMARY

In accordance with embodiments herein, provided is an implantable pulse generator (IPG) for providing electrical pulses to a patient during neurostimulation therapy. The IPG includes a controller for controlling operations of the IPG, and a battery for powering the IPG. The IPG also includes therapeutic stimulation circuitry for generating electrical pulses to stimulate neural tissue of the patient, and internal diagnostic circuitry for digitizing device signals to control operations of the IPG. The internal diagnostic circuitry includes a multi-stage comparator including a pre-amplifier configured to receive an analog input signal and convert the analog input signal into a first analog output signal and second analog output signal. The multi-stage comparator also including a first latch circuit with a first receiving device that receives the first analog output signal, a second receiving device that receives the second analog output signal, and configured to convert the first analog output signal and second analog output signal into a digital output signal.

Optionally, the pre-amplifier, the first receiving device, and the second receiving device are silicon-based. In one aspect, the pre-amplifier, the first receiving device, and the second receiving device are the only silicon-based components of the therapeutic stimulation circuitry. In another aspect, the first latch circuit is a set and reset latch circuit. In one example, the multi-stage comparator includes a second latch circuit coupled to the first latch circuit, and a third latch circuit coupled to the second latch circuit. Optionally, the second latch circuit and third latch circuit each comprise digital devices. In another example, the multi-stage comparator includes successive approximation register logic circuitry to convert the analog input signal into a digital output signal.

Optionally, the multi-stage comparator is on an integrated circuit. In one example, the analog input signal is a high voltage signal, and the digital output signal is a low voltage signal. In one aspect, the digital output is a single bit digital output. In another aspect, the multi-stage comparator further includes timing circuitry to provide timing inputs to the pre-amplifier.

In accordance with embodiments herein, provided is an implantable pulse generator (IPG) for providing electrical pulses to a patient during neurostimulation therapy. The IPG includes a controller for controlling operation of the IPG, and a battery for powering the IPG. The IPG also includes therapeutic stimulation circuitry for generating electrical pulses to stimulate neural tissue of the patient, and sensing circuitry for digitizing patient physiological signals to control therapy operations of the IPG. The sensing circuitry includes a pre-amplifier configured to receive an analog input signal and convert the analog input signal into a first analog output signal and second analog output signal, and a first latch circuit with a first receiving device that receives the first analog output signal, a second receiving device that receives the second analog output signal, and configured to convert the first analog output signal and second analog output signal into a digital output signal.

Optionally, the pre-amplifier, the first receiving device, and the second receiving device are silicon-based. In one aspect, the pre-amplifier, the first receiving device, and the second receiving device are the only silicon-based components of the digitizing circuitry. In another aspect, the first latch circuit is a set and reset latch circuit. In one example, the digitizing circuitry includes successive approximation register logic circuitry to convert the analog input signal into a digital output signal. In another example, the digitizing circuitry is on an integrated circuit. In yet another example, the analog input signal is a high voltage signal, and the digital output signal is a low voltage signal. In one embodiment, the digital output is a single bit digital output. In another embodiment, the digitizing circuitry further includes timing circuitry to provide timing inputs to the pre-amplifier.

### BRIEF DESCRIPTION OF THE DRAWINGS

FIG. 1 depicts a neurostimulation system that is adapted according to an example embodiment and is shown as a high-level functional block diagram.

FIG. 2 depicts a neurostimulation system that is adapted according to an example embodiment and is shown as a high-level functional block diagram.

FIG. 3 depicts a schematic diagram of a portion of a multi-stage comparator according to an example embodiment.

FIG. 4 depicts a flow chart illustrating a method of manufacturing a neurostimulation system.

FIG. 5 depicts a flowchart illustrating a method for discharging electrodes in an implantable medical device.

FIG. 6 depicts a flowchart illustrating a method for monitoring electrodes in an implantable medical device (IMD).

### DETAILED DESCRIPTION

It will be readily understood that the components of the embodiments as generally described and illustrated in the Figures herein, may be arranged and designed in a wide variety of different configurations in addition to the described example embodiments. Thus, the following more detailed description of the example embodiments, as represented in the Figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

### Terms

The term “latch circuit” or “flip flop circuit” as used herein shall refer to a circuit that includes two stable states and can store information. In one example, a latch circuit can be configured to change a state as a result of signals applied to the latch circuit. A latch circuit can be comprised of numerous electronic components, including transistors, resistors, switches, etc. Example latch circuits include set and reset (SR) latch circuits, and latch circuits can be operated utilizing successive approximation register (SAR) logic when utilized within an analog-to-digital converter (ADC).

The term “multi-stage comparator” when used herein refers to a hardware electronic device that converts an analog input into a digital input. The multi-stage comparator can include stages that are latch circuits, pre-amplifiers, or the like, utilized to provide a level shift (e.g. convert a high voltage analog input into a low voltage digital output).

Provided is a neurostimulation system that includes a multi-stage comparator that converts a high voltage analog input into a low voltage digital output utilized to supply signals to the electrodes of the neurostimulation system. The multi-stage comparator includes a pre-amplifier that provides only a first analog output signal and second analog output signal that are received by a first receiving device and a second receiving device, respectively, of a first stage latch circuit. As a result, the pre-amplifier, the first receiving device of the first latch circuit, and the second receiving device of the first latch circuit are the only electronic components of the multi-stage comparator that need to be manufactured from high-voltage material such a silicon. Consequently, the other components of the other stages, including other latch stages, can be made of low-voltage materials that constitute less area, cost, and the like for the integrated circuit having the multi-stage comparator.

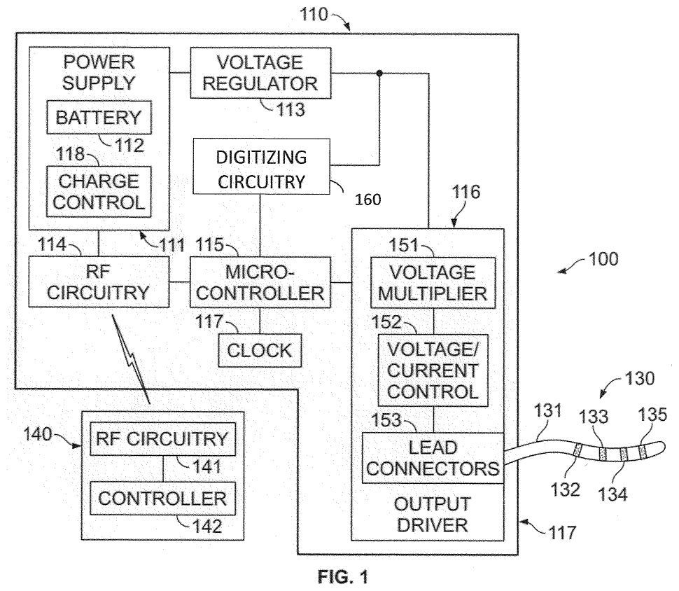

FIG. 1 depicts a neurostimulation system 100 that is adapted according to an example embodiment and is shown as a high-level functional block diagram. Neurostimulation systems are devices that generate electrical pulses and deliver the pulses to neural tissue of a patient to treat a variety of disorders. As noted above, a neurostimulation system 100 may be used to provide DBS therapy for patients with movement disorders. Neurostimulation system 100 may also provide Spinal Cord Stimulation (SCS) in which electrical pulses are delivered to neural tissue of the spinal cord for the purpose of chronic pain control. While a precise understanding of the interaction between the applied electrical energy and the neural tissue is not fully appreciated, it is known that application of an electrical field to spinal neural tissue can effectively inhibit certain types of pain transmitted from regions of the body associated with the stimulated neural tissue to the brain.

Neurostimulation systems generally include a pulse generator and one or more leads. A stimulation lead includes a lead body of insulative material that encloses wire conductors. The distal end of the stimulation lead includes multiple electrodes that are electrically coupled to the wire conductors. The proximal end of the lead body includes multiple terminals (also electrically coupled to the wire conductors) that are adapted to receive electrical pulses. For SCS therapy, the distal end of a respective stimulation lead is implanted within the epidural space to deliver the electrical pulses to the appropriate nerve tissue within the spinal cord. The stimulation leads are then tunneled to another location within the patient's body to be electrically connected with a pulse generator or, alternatively, to an “extension.”

The pulse generator is typically implanted within a subcutaneous pocket created during the implantation procedure. In SCS, the subcutaneous pocket is typically disposed in a lower back region, although subclavicular implantations and lower abdominal implantations are commonly employed for other types of neuromodulation therapies.

The neurostimulation system 100 of the illustrated embodiment includes a generator portion, shown as implantable pulse generator (IPG) 110, for providing a stimulation or energy source, a stimulation portion, shown as lead 130, for application of the stimulus pulse(s), and an optional external controller, shown as programmer/controller 140, to program and/or control IPG 110 via a wireless communications link. IPG 110 may be implanted within a living body (not shown) for providing electrical stimulation from IPG 110 to a selected area of the body, such as a region of the brain or spinal cord, via lead 130. In some embodiments, IPG 110 provides electrical stimulation under control of external programmer/controller 140. It should be appreciated that, although lead 130 is illustrated to provide a stimulation portion of stimulation system 100 and is configured to provide stimulation remotely with respect to the generator portion 110 of neurostimulation system 100, a lead 130 as described herein is intended to encompass a variety of stimulation portion configurations. Furthermore, a lead configuration may include more (e.g., 8, 16, 32, etc.) or fewer (e.g., 1, 2, etc.) electrodes than those represented in the illustrations.

The IPG 110 of the illustrated embodiment includes power supply 111, voltage regulator 113, RF circuitry 114, microcontroller (or microprocessor) 115, therapeutic stimulation circuitry 116, and clock 117, as are described in further detail below. Power supply 111 provides a source of power, such as from battery 112, to other components of IPG 110, as may be regulated by voltage regulator 113. Battery 112 may comprise a non-rechargeable (e.g., single use) battery, a rechargeable battery, a capacitor, and/or like power sources, Charge control 118 provides management for battery 112 and power supply 111 in some embodiments. In some embodiments, the entire IPG 110 device may need to be accessed by a surgical procedure to replace battery 112. In other embodiments, when battery 112 is depleted, it may be recharged after being implanted, for example, inductive coupling and external charging circuits. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

RF circuitry 114 provides data communication between microcontroller 115 and controller 142 in external programmer/controller 140, via RF circuitry 141. It should be appreciated that RF circuitry 114 and/or 141 may be a receiver, a transmitter, and/or transceiver depending upon the communication links desired using far-field and/or near field communication communications. The communication links may be established using suitable communication methods such as inductive wireless communication, low energy BLUETOOTH™ communication, and medical band wireless communication as examples. An example of BLUETOOTH@ communication between an implantable medical device and a programmer device is found, for example, in U.S. Pat. No. 9,894,691, entitled SYSTEMS AND METHODS FOR ESTABLISHING A COMMUNICATION LINK BETWEEN AN IMPLANTABLE MEDICAL DEVICE AND AN EXTERNAL INSTRUMENT, the disclosure of which is incorporated herein by reference.

Microcontroller 115 provides control with respect to the operation of IPG 110, such as in accordance with a program provided thereto by external programmer/controller 140. Software code is typically stored in memory (not shown) of IPG 110 for execution by the microcontroller 115 to control the various components of the device. The software code stored in memory of IPG 110 may support operations of embodiments disclosed herein.

Therapeutic stimulation circuitry 116 generates and delivers pulses to selected ones of electrodes 132-135 on lead body 131 under control of microcontroller 115. For example, voltage multiplier 151 and voltage/current control 152 may be controlled to deliver a constant current pulse of a desired magnitude using selected ones of electrodes 132-135. Clock 117 preferably provides system timing information, such as may be used by microcontroller 115 in controlling system operation, as may be used by voltage multiplier 151 in generating a desired voltage, etcetera.

Lead 130 of the illustrated embodiment includes lead body 131, preferably incorporating a plurality of internal conductors coupled to lead connectors (not shown) to interface with lead connectors 153 of IPG 110. Lead 130 further includes electrodes 132-135, which are preferably coupled to the internal conductors 153. The internal conductors provide electrical connection from individual lead connectors to each of a corresponding one of electrodes 132-235. In the exemplary embodiment the lead 130 is generally configured to transmit one or more electrical signals from IPG 110 for application at, or proximate to, a spinal nerve or peripheral nerve, brain matter, muscle, or other tissue via electrodes 132-135. IPG 110 is capable of controlling the electrical signals by varying signal parameters, such as pulse amplitude, pulse width, pulse frequency, burst frequency, and/or the like in order to deliver a desired therapy or otherwise provide operation as described herein.

Although the embodiment illustrated in FIG. 1 includes four electrodes, it should be appreciated that any number of electrodes, and corresponding conductors, may be utilized according to some embodiments. Moreover, various types, configurations, and shapes of electrodes (and lead connectors) may be used according to some embodiments. An optional lumen (not shown) may extend through the lead 130, such as for use in delivery of chemicals or drugs or to accept a stylet during placement of the lead within the body. Additionally, or alternatively, the lead 130 (stimulation portion) and IPG 110 (generator portion) of stimulation system 100 may comprise a unitary construction, such as that of a microstimulator configuration.

In an embodiment, a programmable neurostimulation system 100 supplies suitable therapy pulses to a patient by enabling a pattern of electrical pulses to be varied across the electrodes 132-135 of a lead or leads 130. Such systems enable electrodes of a connected stimulation lead 130 to be set as an anode(+), as a cathode(−), or to a high-impedance state (OFF). As is well known, negatively charged ions flow away from a cathode toward an anode. Consequently, a range of very simple to very complex electrical fields can be created by defining different electrodes 132-135 in various combinations of (+), (−), and OFF. Of course, in any instance, a functional combination must include at least one anode and at least one cathode. In an embodiment, the case or “can” of the neurostimulation system 100 or IPG 110 may function as an anode. When determining the appropriate electrode configurations, the selection of electrodes 132-135 to function as anodes can often facilitate isolation of the applied electrical field to desired fibers and other neural structures. The selection of an electrode 132-135 to function as an anode at a position adjacent to another electrode functioning as a cathode causes the resulting electron/ion flow to be limited to tissues immediately surrounding the two electrodes. By alternating through the possible anode/cathode combinations, it is possible to gain greater resolution in the stimulation of desired tissue or neural structures.

As mentioned above, programmer/controller 140 provides data communication with IPG 110, such as to provide control (e.g., adjust stimulation settings), provide programming (e.g., alter the electrodes to which stimulation pulses are delivered), etc. Accordingly, programmer/controller 140 of the illustrated embodiment includes RF circuitry 141 for establishing a wireless link with IPG 110, and controller 142 to provide control with respect to IPG 110. Programmer/controller 140 may receive data from IPG 110 that can be displayed to medical personnel or a clinician on a screen (not shown) on programmer/controller 140. Additionally, or alternatively, programmer/controller 140 may provide power to IPG 110, such as via RF transmission by RF circuitry 141. Optionally, however, a separate power controller may be provided for charging the power source 111 within IPG 110.

Additional detail with respect to pulse generation systems and the delivery of stimulation pulses may be found in U.S. Pat. No. 6,609,031, entitled “MULTIPROGRAMMABLE TISSUE STIMULATOR AND METHOD,” the disclosure of which is hereby incorporated herein by reference. Similarly, additional detail with respect to pulse generation systems and the delivery of stimulation pulses may be found in U.S. Pat. No. 7,937,158, entitled “MULTIPROGRAMMABLE TRIAL STIMULATOR,” the disclosure of which is hereby incorporated herein by reference. Also, additional detail with respect to pulse generation system and delivery of stimulation pulses may be found in U.S. Pat. Pub. No. 2021/0402192, entitled “IMPLANTABLE PULSE GENERATOR FOR PROVIDING A NEUROSTIMULATION THERAPY USING COMPLEX IMPEDANCE MEASUREMENTS AND METHODS OF OPERATION,” the disclosure of which is hereby incorporated herein by reference.

The neurostimulation system 100 also includes a digitizing circuitry 160 for digitizing device signals to control operations of the IPG 110. In one example, the digitizing circuitry 160 is internal diagnostic circuitry for digitizing device signals to control operations of the IPG 110. In yet another example, the digitizing circuitry 160 is sensing circuitry for digitizing patient physiological signals to control therapy operation of the IPG. In one example, the sensing circuitry senses bio-markers, and other similar patient physiological signals to control the therapy operation of the IPG. The bio-makers can include action potential for spinal cord stimulation, local field potentials for DBS, or the like. Additional detail with respect to the bio-markers can be found in U.S. Pat. Pub. No. 2019/0255338, entitled “SYSTEM AND METHOD TO CONTROL A NON-PARESTHESIA STIMULATION BASED ON SENSORY ACTION POTENTIALS,” the disclosure of which is hereby incorporated herein by reference.

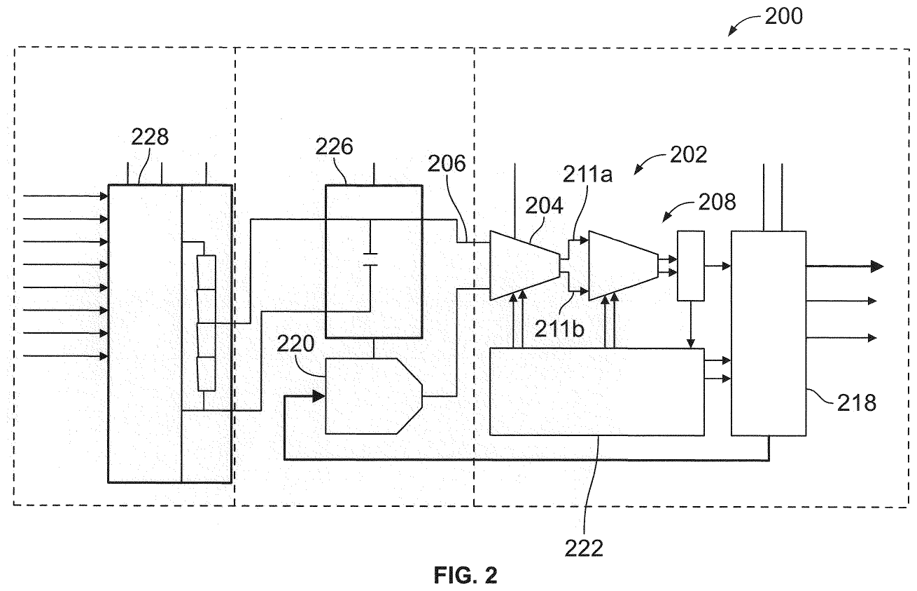

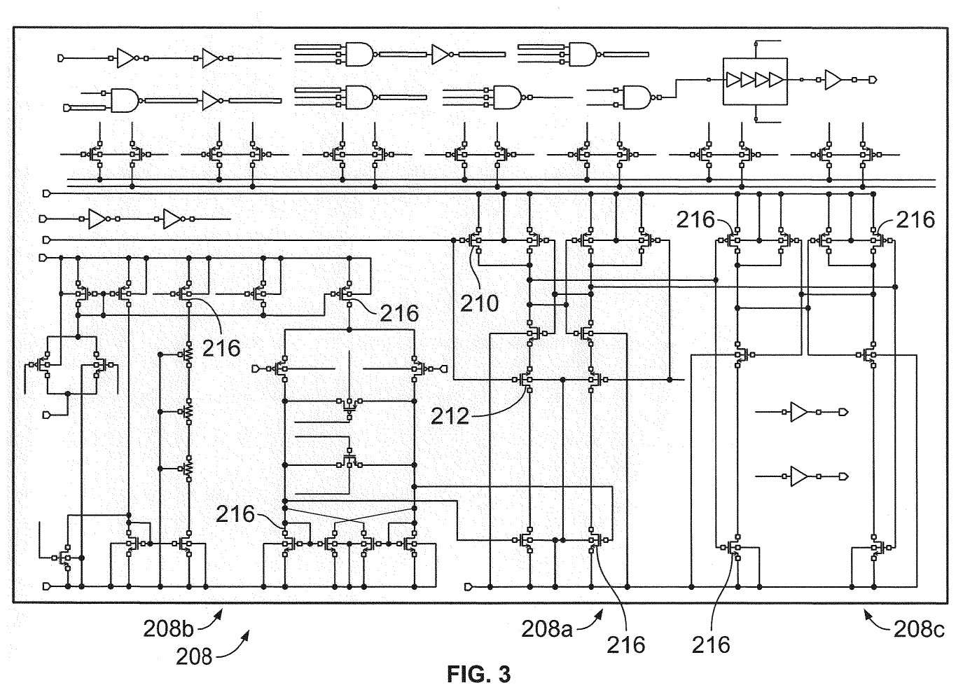

FIG. 2 illustrates a schematic diagram of example digitizing circuitry 200, while FIG. 3 illustrates a portion of the multi-stage comparator 202. In one example the digitizing circuitry 200 of FIG. 2 is the digitizing circuitry 160 of FIG. 1. To this end, the digitizing circuitry 200 in one example is internal diagnostic circuitry configured to control operation of an IPG, whereas in another example, the digitizing circuitry 200 is sensing circuitry that obtains and digitizes patient physiological signals to control therapy operation of the IPG. The digitizing circuitry 200 leverages a mixed-signal integrated circuit process to enable use of a multi-stage comparator architecture (as illustrated in FIG. 3) to transition from a high-voltage analog domain to a low-voltage digital domain.

The digitizing circuitry 200 includes a multi-stage comparator 202 used to achieve high speed and reduce overall offset. In addition, as provided, the multi-stage comparator 202 can also allow a level-shift function to be implemented along with accurate signal comparison. A level-shift function as used herein refers to a circuit used to convert a signal from one voltage domain (e.g. analog) to another (e.g. digital) so that compatibility can be provided between integrated circuits that have different voltage requirements.

The multi-stage comparator 202 includes a first stage pre-amplifier 204 that functions to amplify an analog input signal 206, and to overcome the high offset of the following latch circuits 208. Based on a signal applied, the latch circuit 208, or circuits, can be made to change a state of the signal. The latch circuit 208 can include a first receiving device 210 (FIG. 3) for receiving a first analog output signal 211a of the pre-amplifier 204, and a second receiving device 212 (FIG. 3) for receiving a second analog output signal 211b of the pre-amplifier 204. Use of the pre-amplifier 204 before the latch circuits 208 causes only two signals to be received by two components (e.g. the first receiving device 210 and second receiving device 212) of a single latch circuit 208. In one example, the latch circuits 208 include a first latch circuit 208a, a second latch circuit 208b, and a third latch circuit 208c. In one example, at least one of the first, second, and third latch circuits 208a-c is a set and reset (SR) latch as illustrated in FIG. 3. In this manner, only the first stage pre-amplifier, the first receiving device 210, (that receives the first high-voltage analog output signal 211a) and the second receiving device 212 (that receives the second high-voltage analog output signal 211b) need to be implemented using high-voltage tolerant devices. The remainder of the digitizing circuitry 200, including the rest of the electrical components of the first latch circuit 208a, the electrical components of the second latch circuit 208b, and the electrical components of the third latch circuit 208c, operate at low-voltage. This allows the use of reduced sized digital devices in these latch circuits. In this manner, the first stage (e.g. latches stage) operates on the high voltage power supply while the subsequent stages (e.g. digital devices 216) and downstream digital logic are at a low-voltage digital supply. In one example, high voltage is considered between approximately 80-100V and low voltage is considered between approximately 0-5V.

In one example, the digitizing circuitry 200 also includes successive approximation register (SAR) logic circuitry 218 to provide the analog to digital conversion. In one example, the conversion results in the digital output being only a single bit. In particular, an SAR analog to digital converter utilizes a series of comparisons to determine each bit of a converted result. The analog input signal is tracked by the SAR analog to digital converter, sampled, and held during conversion. The sampled input signal can then be compared to the value of an internal digital to analog converter 220 that adjusts in binary increments to get as close as possible to the sampled value. The digitizing circuitry 200 can also include timing circuitry 222 to provide timing inputs to the pre-amplifier 204 and SAR logic circuitry 218. In one example, the timing circuitry 222 can be self-timed logic circuitry.

In addition, the pre-amplifier 204 receives the analog input signal from a sample 226 provided from a multiplexer 228 (e.g. MUX). In addition, the pre-amplifier 204 additionally receives a feedback input from the SAR logic circuitry 218 via the digital to analog converter 220.

As a result of the digitizing circuitry 200 of FIGS. 2-3, an analog comparison is provided with conversion to a single-bit digital output via a power domain level-shifting that is combined into a single circuit. Only the gain-boosting pre-amplifier 204 and input devices (e.g. first receiving device 210 and second receiving device 212) of the first latch circuit use high-voltage devices. Consequently, the number of high-voltage devices is reduced, reducing the area of the integrated circuit needed for silicon-based, high-voltage devices. Therefore, the area of the integrated circuit is reduced. The digitizing circuitry 200 also allows for direct interfacing to a wider range of analog input signals, including battery voltage and electrode waveforms. In addition, high-voltage device usage to the analog to digital converter input circuits is limited, reducing wear and failures.

The digitizing circuitry 200 also saves power and area by performing the level-shift function as early in the signal chain as possible, and limiting the use of larger high voltage devices. The digitizing circuitry 200 also reduces the need for attenuation of battery voltage and electrode waveform inputs which is a source of non-linearity that must be accounted for during manufacturing trim. As a result, the digitizing circuitry 200 is easier to manufacture, less expensive to manufacture, and is more reliable.

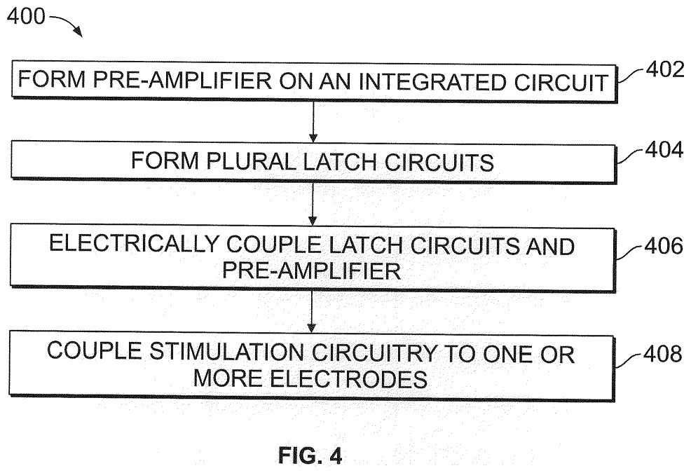

FIG. 4 depicts a block flow diagram of a method 400 for manufacturing an IPG. At 402, pre-amplifier of a multi-stage comparator is formed on an integrated circuit. In one example, the pre-amplifier is the pre-amplifier of FIGS. 2-3. The pre-amplifier can be configured to receive a high voltage analog input signal and convert the analog input signal into a first analog output signal and second analog output signal. In one example, the pre-amplifier is electrically coupled to a sample that receives current from a MUX that provides the analog input signal. By converting the analog input signal into the first analog output signal and second analog output signal, an offset caused by latch circuits can be mitigated.

At 404, plural latch circuits of the multi-stage comparator are formed. The plural latch circuits can include a first latch circuit, a second latch circuit, and a third latch circuit. When formed, the first latch circuit can include a first receiving device, such as a transistor, resistor, switch, or the like, that receives the first analog output signal, and a second receiving device, such as another transistor, resistor, switch, or the like that receives the second analog output signal. In one example, the first receiving device and second receiving device are formed from a silicon based material, whereas all other components in the first latch circuit are formed of a different material. In particular, the first receiving device and second receiving device are the only components of the plural latch circuits that receive the high voltage (between approximately 80-100V) analog output currents (e.g. the first analog output signal and the second analog output signal). At this point a level shift occurs, resulting in the signal converting into a low voltage (between approximately 0-5V) digital current. As a result, the rest of the components (e.g. transistors, resistors, capacitors, etc.) in the first latch circuit, along with all of the components in the second and third latch circuits can be formed of non-silicon based, or low voltage materials.

At 406 the plural latch circuits are electrically coupled to the pre-amplifier. The plural latch circuits may also be coupled to SR latch circuitry, SAR logic circuitry, timing circuitry or the like to form a multi-stage comparator that is formed as a single integrated circuit.

At 408, the digitizing circuitry is electronically coupled to one or more electrodes of a stimulation lead. The electrodes generate and deliver electrical pulses to the patient based on the digital output signal provided by the latch circuits. By providing the pre-amplifier and having only a first and second analog output received by the first and second receiving devices of the first latch circuit, only those components need to be made for high-voltage tolerance. Consequently, the rest of the components of the stimulation circuitry can be made of non-silicon material that is less expensive, and takes up less area of the integrated circuit. Consequently, an improved processing technique is provided.

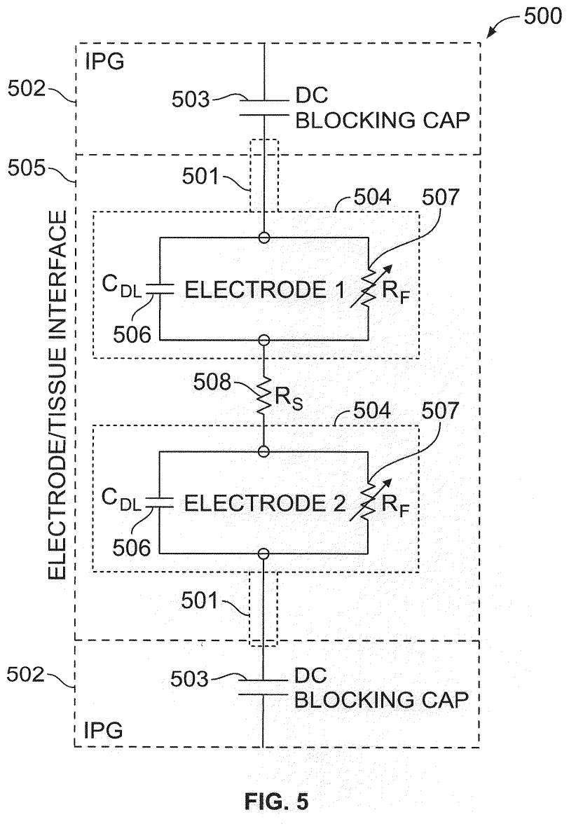

FIG. 5 depicts a model 500 of the electrical interaction between a patient's tissue and stimulation electrodes. One or more stimulation leads 501 are connected to an IPG 502. The stimulation leads 501 are coupled to IPG 502 via DC blocking capacitors 503, which for safety reasons prevent DC current from being applied to the patient's tissue. As a component of IPG 502, the DC blocking capacitors 503 have a known value. In an example embodiment, DC blocking capacitors are 22 .mu.F. Stimulation leads 501 comprise one or more electrodes 504, such as electrodes 132-135 (FIG. 1). When a stimulation therapy waveform is applied to a patient, one electrode 504 may be designated as an anode and the other electrode 504 is designated as a cathode such that charge flows from the anode electrode 504 to the cathode electrode 504. In an embodiment, one electrode may be the metallic case of IPG 502 that functions as an anode.

Model 500 depicts an electrode/tissue interface 505 that represents the electrical characteristics of the physical electrodes that reside in the patient's tissue. The electrode/tissue Interface 505 can be modeled as an RC network for each electrode comprising a parallel capacitor (CDL) 506 in parallel with a variable resistance (RF) 507, which are then in series with at least some portion of the patient tissue resistance (Rs) 508. Capacitor 506 represents the capacitive interface between the electrode and the tissue, which is a double layer (DL) capacitance. At the interface where the physical electrode 504 touches the patient's tissue, electrode 504 acts like a capacitor, which is represented by capacitor (CDL) 506. Where the electrode 504 touches the patient's tissue there are also resistances, which is represented by Faradaic resistance (RF) 507 and some portion of the patient tissue resistance (Rs) 508. Variable resistance 507 accounts for Faradaic conduction across the electrode/tissue interface that is dependent upon the stimulation current density. Resistance (Rs) 508 represents the resistance through and across the patient's tissue between electrodes 504, which is dependent upon the conductivity of the tissue and the effective surface areas of the electrodes. It will be understood that the specific values assigned to the resistances and capacitors in model 500 are dependent upon the type and deployment of electrodes in a particular patient and, therefore, model 500 is unique for each patient and stimulation lead.

The model 500 of an actual electrode/tissue interface 505 In a patient can be determined by taking voltage measurement samples, such as can be taken by IPG 502 across stimulation leads 501 and/or electrodes 504. If only a single voltage measurement is taken, then the electrode/tissue interface can be modelled only as a single resistance, which may or may not be adequate in assessing the impact of the electrodes upon the stimulation delivered by the IPG. However, this is an over-simplification of the load into which the IPG device delivers stimulation. IPG stimulation therapy is not instantaneous, but instead occurs over a period of time, Accordingly, model 500 represents a complex impedance load network for the IPG, which can take time-dependent effects into account, such as caused by capacitances in a load network. By determining model 500 for a patient, IPG 502 can monitor shifts in resistance and capacitance (e.g., R.sub.F, C.sub.DL, or R.sub.S) over time during the life of the patient and device. This provides improved monitoring of lead integrity and patient health and position in some cases.

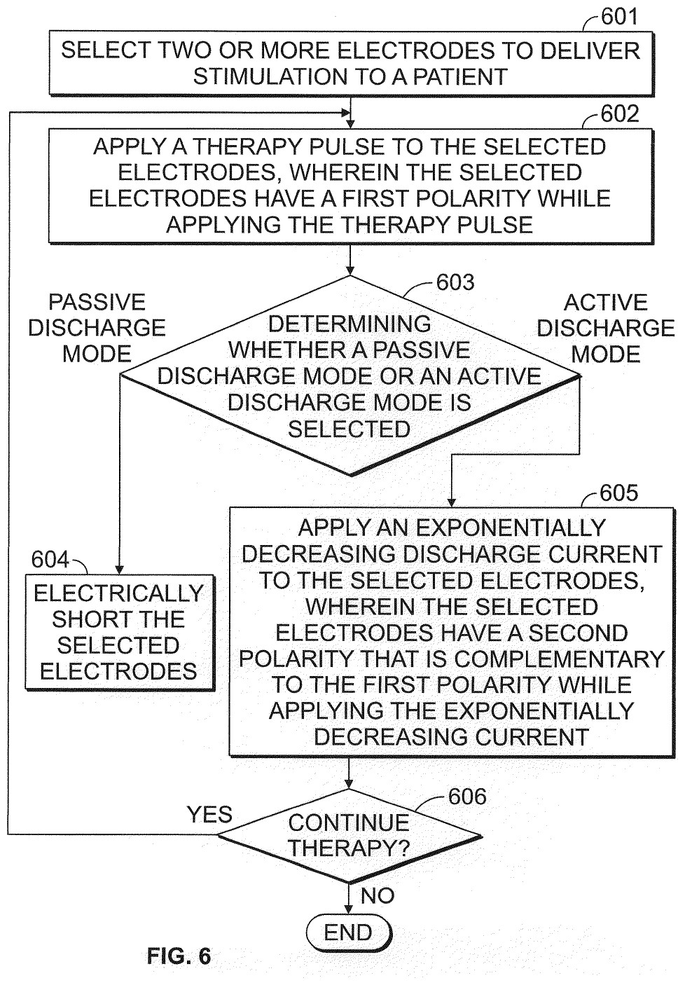

FIG. 6 depicts a flowchart illustrating a method for discharging electrodes in an implantable medical device (IMD). In step 601, one or more electrodes are selected to deliver stimulation to a patient using the IMD. In step 602, a therapy pulse is applied from the IMD to the selected electrodes. The selected electrodes have a first polarity while applying the therapy pulse. In an embodiment, the selected electrodes function as anodes and cathodes in the first polarity. In step 603, a determination is made whether the IMD is operating in a passive discharge mode or an active discharge mode. In one embodiment, the IMD is in communication with an external programming device and receives an active discharge mode command from the external programming device. In another embodiment, the IMD comprises circuitry for identifying the presence of MRI activity or EMI, such as circuitry for detecting electrical and/or magnetic fields. Upon such detection, the IMD may enter an emulated passive discharge mode.

If the IMD Is operating in a passive discharge mode, then in step 604, the selected electrodes are electrically shorted to dissipate stored charge. If the IMD is operating in an active discharge mode, then in step 605, an exponentially decreasing current is applied from the IMD to the selected electrodes having a second polarity which is opposite the first polarity. In an embodiment, the selected electrodes function in a complementary fashion as cathodes and anodes In the second polarity.

In step 606, after completing the passive or exponentially decreasing active discharge in steps 604 or 605, the device determines if therapy should continue. If therapy has been completed, then the process ends. If therapy should continue, then the process returns to step 602 to provide additional therapy pulses.

In one embodiment, the exponentially decreasing discharge current is applied using a programmable current source comprising an error amplifier having a first input coupled to a programmable voltage source and a second input coupled to a programmable resistor. A current at an output of the current regulator is determined by a voltage of the programmable voltage source and a resistance of the programmable resistor. The current output of the current regulator may be decreased in precalculated steps to create the exponentially decreasing discharge current applied to the electrodes.

One or more of the operations described above in connection with the methods may be performed using one or more processors. The different devices in the systems described herein may represent one or more processors, and two or more of these devices may include at least one of the same processors. In one embodiment, the operations described herein may represent actions performed when one or more processors (e.g., of the devices described herein) execute program instructions stored in memory (for example, software stored on a tangible and non-transitory computer readable storage medium, such as a computer hard drive, ROM, RAM, or the like).

The processor(s) may execute a set of instructions that are elements may also store data or other information as desired or needed. The storage element may be in the form of an information source or a physical memory element within the controllers and the controller device. The set of instructions may include various commands that instruct the controllers and the controller device to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

The controller may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. When processor-based, the controller executes program instructions stored in memory to perform the corresponding operations. Additionally, or alternatively, the controllers and the controller device may represent circuits that may be implemented as hardware. The above examples are exemplary only and are thus not intended to limit in any way the definition and/or meaning of the term “controller”.

### Closing Statements

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method, or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the Figures, which illustrate example methods, devices, and program products according to various example embodiments. These program instructions may be provided to a processor of a general purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. An implantable pulse generator (IPG) for providing electrical pulses to a patient during neurostimulation therapy, comprising:
a controller for controlling operations of the IPG;
a battery for powering the IPG;
therapeutic stimulation circuitry for generating electrical pulses to stimulate neural tissue of the patient;
internal diagnostic circuitry for digitizing device signals to control operations of the IPG, wherein the internal diagnostic circuitry comprises:
a multi-stage comparator including a pre-amplifier configured to receive an analog input signal and convert the analog input signal into a first analog output signal and second analog output signal;
the multi-stage comparator also including a first latch circuit with a first receiving device that receives the first analog output signal, a second receiving device that receives the second analog output signal, and configured to convert the first analog output signal and second analog output signal into a digital output signal.

2. The IPG of claim 1, wherein the pre-amplifier, the first receiving device, and the second receiving device are silicon-based.

3. The IPG of claim 2, wherein the pre-amplifier, the first receiving device, and the second receiving device are the only silicon-based components of the therapeutic stimulation circuitry.

4. The IPG of claim 1, wherein the first latch circuit is a set and reset latch circuit.

5. The IPG of claim 1, wherein the multi-stage comparator includes a second latch circuit coupled to the first latch circuit, and a third latch circuit coupled to the second latch circuit.

6. The IPG of claim 5, wherein the second latch circuit and third latch circuit each comprise digital devices.

7. The IPG of claim 1, wherein the multi-stage comparator includes successive approximation register logic circuitry to convert the analog input signal into a digital output signal.

8. The IPG of claim 1, wherein the multi-stage comparator is on an integrated circuit.

9. The IPG of claim 1, wherein the analog input signal is a high voltage signal, and the digital output signal is a low voltage signal.

10. The IPG of claim 1, wherein the digital output is a single bit digital output.

11. The IPG of claim 1, wherein the multi-stage comparator further includes timing circuitry to provide timing inputs to the pre-amplifier.

12. An implantable pulse generator (IPG) for providing electrical pulses to a patient during neurostimulation therapy, comprising:
a controller for controlling operation of the IPG;
a battery for powering the IPG;
therapeutic stimulation circuitry for generating electrical pulses to stimulate neural tissue of the patient; and
sensing circuitry for digitizing patient physiological signals to control therapy operations of the IPG, wherein the sensing circuitry comprises:
pre-amplifier configured to receive an analog input signal and convert the analog input signal into a first analog output signal and second analog output signal;
a first latch circuit with a first receiving device that receives the first analog output signal, a second receiving device that receives the second analog output signal, and configured to convert the first analog output signal and second analog output signal into a digital output signal.

13. The IPG of claim 12, wherein the pre-amplifier, the first receiving device, and the second receiving device are silicon-based.

14. The IPG of claim 13, wherein the pre-amplifier, the first receiving device, and the second receiving device are the only silicon-based components of the digitizing circuitry.

15. The IPG of claim 12, wherein the first latch circuit is a set and reset latch circuit.

16. The IPG of claim 12, wherein the digitizing circuitry includes successive approximation register logic circuitry to convert the analog input signal into a digital output signal.

17. The IPG of claim 12, wherein the digitizing circuitry is on an integrated circuit.

18. The IPG of claim 12, wherein the analog input signal is a high voltage signal, and the digital output signal is a low voltage signal.

19. The IPG of claim 12, wherein the digital output is a single bit digital output.

20. The IPG of claim 12, wherein the digitizing circuitry further includes timing circuitry to provide timing inputs to the pre-amplifier.
